(19) United States
(12) Patent Application Publication
(10) Pub. No.: US 2018/0187069 A1
(43) Pub. Date: Jul. 5, 2018

(54) CHELATING AGENT AND PREPARATION METHOD AND USE THEREOF

(71) Applicant: PetroChina Company Limited, Beijing (CN)

(72) Inventors: Zhiying Deng, Beijing City (CN); Lijun Mu, Beijing City (CN); Hongjun Lu, Beijing City (CN); Bin Yao, Beijing City (CN); Xiaobing Lu, Beijing City (CN); Yong Wang, Beijing City (CN); Zhaojie Song, Beijing City (CN); Lei Sui, Beijing City (CN); Erzhen Wang, Beijing City (CN)

(21) Appl. No.: 15/857,386

(22) Filed: Dec. 28, 2017

(30) Foreign Application Priority Data
Dec. 29, 2016 (CN) 2016112426131

(51) Int. Cl. C09K 8/74; C09K 8/528; E21B 43/28; C07C 229/16; C07C 59/265; C07C 53/08; B01J 19/00

(52) U.S. Cl. CPC C09K 8/74; C09K 8/528; E21B 43/28; C07C 229/16; B01J 2219/00177; C07C 53/08; B01J 19/0013; B01J 2219/00051; C07C 59/265

(57) ABSTRACT

Embodiments of the present invention provide a chelating agent and its preparation method and use. The chelating agent is prepared from the following raw materials in weight percent with respect to 100% of the total weight of the prepared chelating agent: 15% to 30% of an iron ion stabilizing agent, 5% to 12% of dichloroethane, 10% to 20% of ethanol solution, 10% to 20% of sodium hydroxide, 5% to 10% of carbon disulfide, 1.5% to 4.5% of an adjuster, and water as balance. The raw materials for the chelating agent according to the present invention are widely available from a variety of sources, and are less expensive. The chelating agent according to the present invention has a stable performance and a long lifetime, can be stored and use for a long time, and is highly compatible with acidizing acid liquid systems.

### TECHNICAL FIELD

Embodiments of the present invention relate to the field of oil well development, particularly to the field of acidizing and unplugging of water-injection wells, and more particularly to a chelating agent and the preparation method and use thereof.

### BACKGROUND

During acidizing treatment of an oil field, highly concentrated acid solution dissolves pipelines, devices and underground clay minerals and produces precipitates during stirring and pump-injection of the acid solution, and thus it is necessary to add a chelating agent to the acid solution to inhibit formation of precipitates.

Currently widely employed chelating agents mainly include lactic acid, acetic acid, ethylenediaminetetraacetic acid (EDTA), dihydroxymaleic acid, gluconic acid, polyphosphate, sodium isoascorbate, and the like. These chelating agents suffer from problems such as a low yield, poor selectivity, and high cost in their production process, and have drawbacks such as poor temperature resistance, compatibility, stability and inhibiting performance in treatment operations at acid-injection sites. As a result, conventional acidizing processes require use of three type of fluids, i.e. a preflush fluid, a treating fluid, and an overflush liquid, and post-treatment fluid flowback is necessary, which complicate the treatment process.

### SUMMARY OF INVENTION

An object of the present invention is to provide a chelating agent which has a great ability to inhibit precipitation, good stability, and a simple preparation process, can be directly added to an acid solution system without compromising the effect of the acid solution, and can adsorb metal ions.

Another object of the present invention is to provide a preparation method of the chelating agent.

Yet another object of the present invention is to provide use of the chelating agent.

To meet the above objects, embodiments of the present invention provide a chelating agent, which is prepared from the following raw materials in weight percent with respect to the total weight (100%) of the prepared chelating agent:

| | |
|---|---|
| Iron ion stabilizing agent | 15% to 30% |
| Dichloroethane | 5% to 12% |
| Solvent | 10% to 20% |
| Sodium hydroxide | 10% to 20% |
| Carbon disulfide | 5% to 10% |
| Adjuster | 1.5% to 4.5% |
| Water | Balance. |

According to some specific embodiments of the present invention, the chelating agent is prepared from the following raw materials in weight percent:

| | |
|---|---|
| Iron ion stabilizing agent | 30% |
| Dichloroethane | 12% |
| Solvent | 20% |
| Sodium hydroxide | 20% |
| Carbon disulfide | 10% |
| Adjuster | 3% |
| Water | Balance. |

According to some specific embodiments of the present invention, the iron ion stabilizing agent is an aqueous solution of EDTA and nitrilotriacetic acid (NTA), wherein the mass ratio of EDTA:NTA is (1 to 1.5):(2 to 2.5).

According to some specific embodiments of the present invention, the mass ratio of EDTA:NTA is 1:2.

According to some specific embodiments of the present invention, in the aqueous solution of EDTA and NTA, the mass ratio of EDTA:NTA:water is (1 to 1.5):(2 to 2.5):(5 to 6).

According to some specific embodiments of the present invention, in the aqueous solution of EDTA and NTA, the mass ratio of EDTA:NTA:water is 1:2:5.

According to some specific embodiments of the present invention, the solvent is ethanol or toluene.

According to some specific embodiments of the present invention, the adjuster is a mixed solution of a 36 wt % citric acid aqueous solution and a 64 wt % acetic acid aqueous solution, wherein the mass ratio of the citric acid aqueous solution to the acetic acid aqueous solution is (2 to 3):(1 to 1.5).

According to some specific embodiments of the present invention, the mass ratio of the citric acid aqueous solution to the acetic acid aqueous solution is 2:1.

It is to be understood that the adjuster is a mixed solution obtained by mixing a 36 wt % citric acid aqueous solution and a 64 wt % acetic acid aqueous solution.

In another aspect, embodiments of the present invention provide a method for preparing the chelating agent, comprising the steps of:

weighing out each of the raw materials,
adding dichloroethane dropwise to a mixed solution of the iron ion stabilizing agent and the solvent to carry out a reaction;
after the reaction is completed, adding water and sodium hydroxide, and dropwise adding carbon disulfide to carry out a reaction, and
after the reaction is completed, adjusting the pH of the reaction solution to 2 to 5 with the adjuster, followed by a reaction under stirring, to obtain the chelating agent.

According to some specific embodiments of the present invention, the method comprises the steps of:

weighing out each of the raw materials,
adding dichloroethane dropwise to a mixed solution of the iron ion stabilizing agent and the solvent at a temperature of 60° C. to 70° C. to carry out a reaction;
after the reaction is completed, cooling the reaction solution, adding water and sodium hydroxide, and dropwise adding carbon disulfide to carry out a reaction at room temperature for 1 to 4 h,
then elevating the temperature to 30° C. to 60° C. to carry out a reaction; and
after the reaction is completed, cooling the reaction solution and adjusting the pH of the reaction solution to 2 to 5 with the adjuster, followed by a reaction under stirring, to obtain the chelating agent.

According to some specific embodiments of the present invention, the method comprises the steps of:

weighing out each of the raw materials,
adding dichloroethane dropwise to a mixed solution of the iron ion stabilizing agent and the solvent at a temperature of 60° C. to 70° C., to carry out a reaction for 30 to 90 min;
after the reaction is completed, cooling the reaction solution, adding water and sodium hydroxide, and dropwise adding carbon disulfide to carry out a reaction at room temperature for 1 to 4 h,
then elevating the temperature to 30° C. to 60° C. to carry out a reaction for 30 to 90 min; and
after the reaction is completed, cooling the reaction solution and adjusting the pH of the reaction solution to 2 to 5 with the adjuster, followed by a reaction for 10 to 30 min under stirring, to obtain the chelating agent.

According to some specific embodiments of the present invention, the iron ion stabilizing agent is an aqueous solution of EDTA and NTA, and the method comprises: weighing out each of the raw materials, adding NTA and the solvent sequentially to EDTA, and then adding dichloroethane dropwise to a mixed solution of the iron ion stabilizing agent and the solvent to carry out a reaction.

According to some specific embodiments of the present invention, the iron ion stabilizing agent comprises EDTA and NTA, and is prepared by the steps of: adding EDTA into a container equipped with an electric stirrer and a reflux condenser, and then adding NTA and the solvent sequentially.

In another aspect, embodiments of the present invention provide use of the chelating agent in an acidizing acid liquid system.

According to some specific embodiments of the present invention, the chelating agent is added directly to an acidizing acid liquid system.

According to some specific embodiments of the present invention, the chelating agent is used for acidizing and unplugging of a water-injection well.

In summary, embodiments of the present invention provide a chelating agent and its preparation method and use. The chelating agent according to the present invention has the following advantages:

1) the raw materials for the chelating agent according to the present invention are widely available from a variety of sources, and are less expensive;
2) the chelating agent according to the present invention has a stable performance and a long lifetime, can be stored and use for a long time, and is highly compatible with acidizing acid liquid systems;
3) the chelating agent according to the present invention can efficiently chelate metal ions such as Ca²⁺, Al³⁺ and Fe³⁺, to prevent formation of secondary or tertiary precipitates such as fluoroaluminates and fluorosilicates;
4) the chelating agent according to the present invention has higher precipitation-inhibiting ability from below 50% to over 70%, as compared to conventional chelating agents;
5) the chelating agent according to the present invention is simple to prepare, and reacts quickly; and
6) the chelating agent according to the present invention can be used directly upon dilution at acidizing sites with an acid liquid system, and imparts the acid liquid system triple functions of a preflush fluid, a treating fluid and an overflush liquid, thereby simplifying the treatment process and improving the treatment effectiveness.

### DETAILED DESCRIPTION OF INVENTION

The implementation of the present invention and the beneficial effects produced thereby will be described in detail hereinafter with reference to the Examples which are intended to help readers better understand the spirit and features of the present invention, but do not limit the implementable scope of the present invention.

### Example 1

Example 1 provides a chelating agent consisting of the following raw materials in weight percent: 15% to 30% of an iron ion stabilizing agent, 5% to 12% of dichloroethane, 10% to 20% of ethanol, 10% to 20% of sodium hydroxide, 5% to 10% of carbon disulfide, 1.5% to 4.5% of an adjuster, and water as balance.

The iron ion stabilizing agent can chelate metal ions such as Ca²⁺, Al³⁺ and Fe³⁺, to prevent formation of secondary or tertiary precipitates. The solvent enables a homogenous reaction between the iron ion stabilizing agent and dichloroethane, and is a chemically or technically pure product commercially available. The dichloroethane represents 50% to 100% of the mass of the aqueous phase each time, and reacts with the iron ion stabilizing agent to improve its chelating ability. The sodium hydroxide is a chemically or technically pure product commercially available, and is used for increasing the reaction rate. The carbon disulfide is an industrial product serving as an analytical-grade solvent and a reactant. The adjuster is used to adjust the pH and temperature resistance of the chelating agent.

The iron ion stabilizing agent can efficiently chelate metal ions such as Ca²⁺, Al³⁺ and Fe³⁺ and prevent formation of secondary or tertiary precipitates, so that the in-line acid liquid can replace the three-step operation using a preflush fluid, a treating fluid and an overflush liquid in conventional acidizing treatment, thereby simplifying the acidizing treatment process, saving the time and cost of the treatment, lowering the safety risk, reducing the workload, and improving the treatment effectiveness.

### Example 2

In accordance with Example 1, the iron ion stabilizing agent is a solution of EDTA and NTA, wherein the mass ratio of EDTA:NTA is (1 to 1.5):(2 to 2.5). Preferably, the mass ratio of EDTA:NTA is 1:2.

### Example 3

In accordance with Example 1, the adjuster is a solution of a 36 wt % citric acid solution and a 64 wt % acetic acid solution in a mass ratio of (2 to 3):(1 to 1.5). Preferably, the mass ratio of the citric acid solution to the acetic acid solution is 2:1.

### Example 4

Example 4 provides a chelating agent consisting of the following raw materials in weight percent: 5% of EDTA, 10% of NTA, 6% of dichloroethane, 10% of toluene, 10% of sodium hydroxide, 5.3% of carbon disulfide, 1% of citric acid, 0.5% of acetic acid, and water as balance.

The chelating ability of the chelating agent for metal ions was tested and shown in Table 1 below.

TABLE 1
Chelating ability of the chelating agent for metal ions

| Ca²⁺ Chelating | Al³⁺ Chelating | Fe³⁺ Chelating | Mg³⁺ Chelating |
|---|---|---|---|
| ability/ | ability/ | ability/ | ability/ |
| (mg · L⁻¹) | (mg · L⁻¹) | (mg · L⁻¹) | (mg · L⁻¹) |
| 260 | 220 | 445 | 162 |

### Example 5

Example 5 provides a chelating agent consisting of the following raw materials in weight percent: 6% of EDTA, 12% of NTA, 7.2% of dichloroethane, 12% of ethanol, 12% of sodium hydroxide, 6.3% of carbon disulfide, 1.2% of citric acid, 0.6% of acetic acid, and water as balance.

The chelating ability of the chelating agent for metal ions was tested and is shown in Table 2 below.

TABLE 2
Chelating ability of the chelating agent for metal ions

| Ca²⁺ Chelating | Al³⁺ Chelating | Fe³⁺ Chelating | Mg³⁺ Chelating |
|---|---|---|---|
| ability/ | ability/ | ability/ | ability/ |
| (mg · L⁻¹) | (mg · L⁻¹) | (mg · L⁻¹) | (mg · L⁻¹) |
| 270 | 230 | 456 | 170 |

### Example 6

Example 6 provides a chelating agent consisting of the following raw materials in weight percent: 5.5% of EDTA, 11% of NTA, 6.6% of dichloroethane, 11% of toluene, 11% of sodium hydroxide, 5.8% of carbon disulfide, 1.1% of a citric acid solution, 0.55% of acetic acid, and water as balance.

The chelating ability of the chelating agent for metal ions was tested and shown in Table 3 below.

TABLE 3
Chelating ability of the chelating agent for metal ions

| Ca²⁺ Chelating | Al³⁺ Chelating | Fe³⁺ Chelating | Mg³⁺ Chelating |
|---|---|---|---|
| ability/ | ability/ | ability/ | ability/ |
| (mg · L⁻¹) | (mg · L⁻¹) | (mg · L⁻¹) | (mg · L⁻¹) |
| 262 | 223 | 451 | 168 |

### Example 7

Example 7 provides a chelating agent consisting of the following raw materials in weight percent: 10% of EDTA, 20% of NTA, 12% of dichloroethane, 20% of toluene, 20% of sodium hydroxide, 10% of carbon disulfide, 2% of citric acid, 1% of acetic acid, and water as balance.

The chelating ability of the chelating agent for metal ions was tested and shown in Table 4 below.

TABLE 4
Chelating ability of the chelating agent for metal ions

| Ca²⁺ Chelating | Al³⁺ Chelating | Fe³⁺ Chelating | Mg³⁺ Chelating |
|---|---|---|---|
| ability/ | ability/ | ability/ | ability/ |
| (mg · L⁻¹) | (mg · L⁻¹) | (mg · L⁻¹) | (mg · L⁻¹) |
| 285 | 238 | 460 | 182 |

### Example 8

Example 8 provides a chelating agent consisting of the following raw materials in weight percent: 8% of EDTA, 16% of NTA, 9.6% of dichloroethane, 16% of ethanol, 16% of sodium hydroxide, 8% of carbon disulfide, 1.6% of citric acid, 0.8% of acetic acid, and water as balance.

The chelating ability of the chelating agent for metal ions was tested and is shown in Table 5 below.

TABLE 5
Chelating ability of the chelating agent for metal ions

| Ca²⁺ Chelating | Al³⁺ Chelating | Fe³⁺ Chelating | Mg³⁺ Chelating |
|---|---|---|---|
| ability/ | ability/ | ability/ | ability/ |
| (mg · L⁻¹) | (mg · L⁻¹) | (mg · L⁻¹) | (mg · L⁻¹) |
| 275 | 230 | 456 | 179 |

### Example 9

Example 9 provides a method for preparing the chelating agent, comprising the steps of: weighing out each of the components according to their specified amounts; adding the iron ion stabilizing agent and the solvent to a container and placing the container into a thermostatic waterbath at 60° C. to 70° C.; adding dicloroethane dropwise thereto under stirring to carry out a reaction for 30 to 90 min; after cooling, adding water and sodium hydroxide in their specified amounts, respectively, and dropwise adding carbon disulfide in molar equivalent to the sodium hydroxide, to carry out a reaction at room temperature for 1 to 4 h; then elevating the temperature to 30° C. to 60° C. to carry out a reaction for 30 to 90 min; after cooling, adjusting the pH of the reaction solution to 2 to 5 with the adjuster, followed by a reaction for 10 to 30 min under continuous stirring, to obtain the chelating agent after the reaction is completed.

### Example 10

Example 10 provides use of the chelating agent in an acidizing acid liquid system, wherein the chelating agent is added directly to the acidizing acid liquid system.

The acidizing acid liquid system to which the chelating agent has been added is injected by a continuous-injection acidizing technique, to perform acidizing treatment of a water-injection well. The continuous-injection acidizing technique is a known technique in the art, the detailed description of which is omitted here.

The acidizing acid liquid system to which the chelating agent has been added chelates and extracts metal ions such as Ca²⁺, Al³⁺ and Fe³⁺, which are mainly disposed in water-injection wells.

### Example 11

Example 11 provides a method for preparing the chelating agent, comprising the steps of: weighing out each of the components in the prescribed amounts according to Example 7; placing the specified amount of EDTA into a container equipped with an electric stirrer and a reflux condenser, then adding the specified amounts of NTA and the solvent separately; then placing the container into a thermostatic waterbath at 70° C.; adding dicloroethane dropwise thereto under stirring to carry out a reaction for 60 min; after cooling, adding the specified amounts of water and sodium hydroxide separately, and dropwise adding carbon disulfide slowly in molar equivalent to the sodium hydroxide, to carry out a reaction at room temperature for 3 h; then elevating the temperature to 40° C. to carry out a reaction for 60 min; after cooling, adjusting the pH of the reaction solution to 2 to 5 with the specified amounts of citric acid and acetic acid, followed by a reaction for 20 min under continuous stirring, to obtain the chelating agent after the reaction is completed.

The chelating agent prepared according to Example 11 was tested in an acidizing treatment process in the Changqing Oil Field. The evaluation results of the inhibitory abilities of an acid liquid (mud acid and multi-hydrogen acid) without the chelating agent highly capable of inhibiting precipitation, and an acid liquid to which the chelating agent was added at 3% by weight (in-line injected acid), are compared and shown in Table 6.

TABLE 6
Comparison of inhibitory ability between mud acid and multi-hydrogen
acid to which the chelating acid is not added and an acid liquid to which
the chelating agent has been added.

| | Inhibition of | Inhibition of | Inhibition of | Inhibition of |
|---|---|---|---|---|
| | CaF₂ | Na₂SiF₆ | Al(OH)₃ | Fe(OH)₃ |
| Acid liquid | (%) | (%) | (%) | (%) |
| Mud acid | — | — | — | — |
| Multi- | 60.72% | 31.45% | 40.44% | 60.40% |
| hydrogen | | | | |
| acid | | | | |
| In-line | 93.71% | 67.81% | 58.81% | 77.32% |
| injected | | | | |
| acid | | | | |

The evaluation results indicate that the chelating agent for the in-line injected acid has a strong ability to inhibit precipitation, avoids secondary precipitation, lays a fundamental basis for treatment without a flowback operation, and is highly valuable for practical application, specifically in that

1) the chelating agent can efficiently chelate metal ions such as Ca²⁺, Al³⁺ and Fe³⁺, and effectively prevents formation of secondary or tertiary precipitates such as fluoroaluminates and fluorosilicates;
2) the chelating agent is very compatible with the acid fluid, can be directly used upon dilution, and does not require on-site preparation; and
3) the acid liquid to which the chelating agent has been added can replace the three-step operation of a preflush fluid, a treating fluid and an overflush liquid in conventional acidizing treatment, without affecting the corrosive effect of mud acid on reservoir rocks.

It is apparent that various modifications and improvements of the present invention can be made by a person skilled in the art without departing from the spirit and scope of the present invention. Therefore, as long as these modifications and improvements of the present invention are within the scope of the pending claims and their equivalents, these modifications and improvements are intended to be included in the purview of the present invention.

1. A chelating agent prepared from the following raw materials in weight percent with respect to 100% of the total weight of the prepared chelating agent:

| | |
|---|---|
| Iron ion stabilizing agent | 15% to 30% |
| Dichloroethane | 5% to 12% |
| Solvent | 10% to 20% |
| Sodium hydroxide | 10% to 20% |
| Carbon disulfide | 5% to 10% |
| Adjuster | 1.5% to 4.5% |
| Water | Balance. |

2. The chelating agent according to claim 1, prepared from the following raw materials in weight percent:

| | |
|---|---|
| Iron ion stabilizing agent | 30% |
| Dichloroethane | 12% |
| Solvent | 20% |
| Sodium hydroxide | 20% |
| Carbon disulfide | 10% |
| Adjuster | 3% |
| Water | Balance. |

3. The chelating agent according to claim 1, wherein the iron ion stabilizing agent is an aqueous solution formulated with ethylenediaminetetraacetic acid (EDTA) and nitrilotriacetic acid (NTA),
wherein the mass ratio of EDTA:NTA is (1 to 1.5):(2 to 2.5), preferably 1:2; and wherein the mass ratio of EDTA:NTA:water is preferably (1 to 1.5):(2 to 2.5):(5 to 6), more preferably 1:2:5.

4. The chelating agent according to claim 1, wherein the solvent is ethanol or toluene.

5. The chelating agent according to claim 1, wherein the adjuster is a mixed solution of a 36 wt % citric acid aqueous solution and a 64 wt % acetic acid aqueous solution, wherein the mass ratio of the citric acid aqueous solution to the acetic acid aqueous solution is (2 to 3):(1 to 1.5), preferably 2:1.

6. A method for preparing the chelating agent according to claim 1, comprising the steps of:
weighing out each of the raw materials,
adding dichloroethane dropwise to a mixed solution of the iron ion stabilizing agent and the solvent to carry out a reaction;
after the reaction is completed, adding water and sodium hydroxide, and dropwise adding carbon disulfide to carry out a reaction; and
after the reaction is completed, adjusting the pH of the reaction solution to 2 to 5 with the adjuster, followed by a reaction under stirring, to obtain the chelating agent.

7. The method according to claim 6, comprising the steps of:
weighing out each of the raw materials,
adding dichloroethane dropwise to a mixed solution of the iron ion stabilizing agent and the solvent at a temperature of 60° C. to 70° C. to carry out a reaction;
after the reaction is completed, cooling the reaction solution, adding water and sodium hydroxide, and dropwise adding carbon disulfide to carry out a reaction at room temperature for 1 to 4 h;
then elevating the temperature to 30° C. to 60° C. to carry out a reaction; and
after the reaction is completed, cooling the reaction solution and adjusting the pH of the reaction solution to 2 to 5 with the adjuster, followed by a reaction under stirring, to obtain the chelating agent.

8. The method according to claim 7, comprising the steps of:
weighing out each of the raw materials,
adding dichloroethane dropwise to a mixed solution of the iron ion stabilizing agent and the solvent at a temperature of 60° C. to 70° C., to carry out a reaction for 30 to 90 min; after the reaction is completed, cooling the reaction solution, adding water and sodium hydroxide, and dropwise adding carbon disulfide to carry out a reaction at room temperature for 1 to 4 h;
then elevating the temperature to 30° C. to 60° C. to carry out a reaction for 30 to 90 min; and
after the reaction is completed, cooling the reaction solution and adjusting the pH of the reaction solution to 2 to 5 with the adjuster, followed by a reaction for 10 to 30 min under stirring, to obtain the chelating agent.

9. The method according to claim 6, wherein the iron ion stabilizing agent is an aqueous solution formulated with EDTA and NTA, and the method comprises: weighing out each of the raw materials, adding NTA and the solvent sequentially to EDTA, and then adding dichloroethane dropwise to a mixed solution of the iron ion stabilizing agent and the solvent to carry out a reaction.

10. Use of the chelating agent according to claim 1 in an acidizing acid liquid system.
